(19) United States
(12) Patent Application Publication
(10) Pub. No.: US 2015/0027451 A1
(43) Pub. Date: Jan. 29, 2015

(54) ANTI-ROTATION WEDGE

(71) Applicant: SKIL-CARE CORPORATION, Yonkers, NY (US)

(72) Inventor: Francis McNamee, Yonkers, NY (US)

(73) Assignee: SKIL-CARE CORPORATION, Yonkers, NY (US)

(21) Appl. No.: 14/266,036

(22) Filed: Apr. 30, 2014

Related U.S. Application Data
(63) Continuation-in-part of application No. 29/461,557, filed on Jul. 24, 2013, now Pat. No. D718456.

(51) Int. Cl. A61F 5/37

(52) U.S. Cl. CPC A61F 5/37
USPC 128/882

(57) ABSTRACT

Disclosed is a device for stabilizing a limb, e.g., to which a health care device is secured, and providing for anti-rotation thereof in a health care setting. In an embodiment, the device includes a base section, and two side sections attached to the base section and extending therefrom to define a trough-shaped channel or a rectangularly-shaped channel. Each side section includes a plurality of discontinuities with edges extending longitudinally along an interior side thereof. The discontinuities provide engagement with a limb or health care device within the channel to resist bi-lateral rotation thereof in the channel. A similar embodiment includes one side section and provides unilateral anti-rotation resistance.

### CROSS REFERENCE TO RELATED APPLICATION

This application is a continuation-in-part of U.S. patent application Ser. No. 29/461,557 filed Jul. 24, 2013, which is incorporated by reference herein in its entirety.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyrights whatsoever.

### BACKGROUND

This application relates generally to healthcare products used to prevent rotation of a reclining or seated person's limb in institutional or home health care settings. More specifically, this application relates to anti-rotation or stabilization devices or “wedges” for use in combination with a health care device worn by a reclining or seated person, for example, a heel boot configured to off-load a patient's heel from a bed.

Patients in a health care setting, such as hospitals and nursing homes, acute and sub-acute rehabilitation centers, hospice, and other health care settings, including in the home (hereinafter simply a “health care setting”) often suffer from health conditions or ailments that prevent, or minimize, patient movement. At times, the health or condition of a patient may require that the patient be confined to a bed, a gurney, or other piece of medical equipment designed for non-ambulatory patients such as a reclining chair for prolonged periods of time. For ease of description, and without limitation, unless the context indicates otherwise, the term “bed” encompasses chairs, recliners, gurneys, etc.

For example, a patient in poor health, or unable to walk under his or her own power, or a post-surgery patient may need to remain in bed during a recovery or healing period. Even in situations where a patient can be transported or moved, such for testing or rehabilitation services, the patient may spend a majority of any given day reclining and/or seated.

Bed confinement, even for shorter periods of time, may cause patients to develop health conditions, whether resulting from prolonged contact with the bed, or inactivity, or may exacerbate existing conditions. For example, patients may suffer from pressure ulcers, plantar flexion (or “foot drop” due to weakness, nerve damage, or paralysis of muscles), tissue injury, deep tissue injury, foot splaying, strains, or other conditions relating to the foot, heel, lower leg, limbs, and other areas of the body. Such conditions may increase a patient's discomfort, fatigue, recovery time, or lead to other conditions such as infection or conditions requiring long-term care. In some cases, conditions resulting from confinement to a bed may lead to a drastic increase in hospitalization time, recovery time, or rehabilitation time, as well as patient care costs.

As one example, pressure ulcers (or “decubitus ulcers” or “pressure sores” or “bedsores”), which may be a localized injury to the skin and/or underlying tissue over a bony prominence as a result of pressure, are often particularly troublesome and may affect a significant percentage of patients in any given institutional or home health care setting. Heel pressure ulcers, in particular, often result from the nature of the heel anatomy, i.e., a relatively small bony surface area that can be exposed to significant pressure transfer against thin subcutaneous tissue when a patient is confined to a bed for a prolonged period of time. Such pressure ulcers may be painful and difficult to treat.

One solution to prevent or treat heel pressure ulcers is a heel protector boot (hereinafter “heel boot”), which off-loads, elevates, or “floats” a patient's heel from a bed by suspending it over an air cavity, or otherwise lifts a patient's heel from a supporting surface, such as a bed surface, and generally reduces, or redistributes pressure on the heel area. Heel boots may also provide for increased ventilation and air circulation around the heel and foot areas while a patient is in bed, and may also promote perfusion, i.e., the delivery of blood to biological tissue, in cases of heel ulcers. Two exemplary heel boots are sold by Skil-Care™ Corporation as the Super Soft Heel Protector™ and the Heel Float™.

While heel boots and other devices may be successful in preventing or treating pressure ulcers, plantar flexion, or other conditions in health care settings, such devices are prone to rotation caused, e.g., by patient movement. In the case of heel boots, for example, a patient's lower leg, heel, and foot while in a boot may laterally rotate or “roll,” with rotation of the boot, on a supporting surface such on a bed. Lateral rotation of the foot or leg in health care settings may contribute to conditions relating to external or internal hip rotation or to conditions relating to the peroneal nerve. In other cases, lateral rotation may exacerbate existing patient conditions relating to the foot, leg, heel, toes, or limbs in general. And yet in other cases, lateral rotation may cause or increase general patient discomfort, which may lead to patient non-compliance with pressure offloading devices or pressure reduction protocols.

### SUMMARY

According to some embodiments, a stabilizing device, adapted to stabilize a limb or health care device worn on the limb, comprises a base section and one or more side sections attached to the base section and extending therefrom to define a one- or two-sided channel for receiving the limb or health care device. The base and side section(s) define a channel exterior that resists rotation of the stabilizing device when contacting a bed or chair surface or other supporting surface. In such embodiments, the base section defines the bottom of the channel and each side section defines a side of the channel, and the channel is adapted to receive the limb or health care device on the channel bottom contacting the bottom section with a side section or sections contacting the limb or device. At least one of the side sections includes at least one surface discontinuity in the interior side thereof adapted to engage a limb or device received in the channel and resist rotation thereof relative to the stabilizing device. The discontinuity provides a griping or engaging function of the stabilizing device relative to the limb or health care device. Thus, the stabilizing device resists rotation thereof relative to a supporting surface, and resists rotation of a limb or health care device therein.

According to various embodiments, the side section(s) may be wedge-shaped or block-shaped, or may have other shapes. For example, a side section comprises an interior side facing the channel interior that in various embodiments may be angled or tapered relative to the bottom section (i.e., wedge-shaped), or generally perpendicular to the bottom section (i.e., block-shaped), or otherwise contoured or shaped.

According to some embodiments, the bottom section comprises a sheet and in some embodiments comprises a flat interior and exterior surface (relative to a channel).

According to various embodiments, a stabilizing device comprising one side section (a one-sided channel device) may provide unilateral stabilization, i.e., the device may resist limb rotation in one direction while allowing rotation in another direction, while a stabilizing device comprising two side sections (a two-sided channel device) may provide bilateral stabilization, i.e., the device may resist limb rotation in two directions.

According to various embodiments, the discontinuities may take various shapes and configurations. For example, a discontinuity may comprise a discrete structure such as a spike or tab of limited width, which presents an edge or edges, or a tip or point or tips or points, or a more continuous structure which presents an edge a more continuous edge. According to some embodiments, such structure in the surface of the interior side is adapted to engage or grip a limb or health care device and thereby resist rotation thereof. According to various embodiments, an edge may be provided by a recess, or a slot, or a projection, or a tab, or a step. A plurality of edges may be so provided, or by terraces in the interior side of a side section.

According to some embodiments, an edge extends longitudinally across the interior side of a side section (longitudinally being relative to the channel or a limb received in the stabilizing device). An edge may extend along the interior of a side section for a distance effective for the edge, or a plurality of edges, to achieve the engaging or gripping function. According to some embodiments, an edge may extend for a minor or major portion of, or for the entire extent, of a side portion, depending upon the configuration of the edge(s) and the number of edges in a side or sides. An edge may also be referred to as a step

In some embodiments, the stabilizing device is configured and/or sized to permit easy separation of a limb or health care device from the stabilizing device while at the same time the stabilizing device performs its anti-rotation function. For example, lifting a limb while in the stabilizing device will result in such separation, per se, or with minor assistance.

According to some embodiments, the stabilizing device is a unitary structure, e.g., a homogeneous structure. In some embodiments, the stabilizing device is made of foam of a density, and is of a size, suitable for the device to perform its anti-rotation functions, e.g., similar to those of the “Bilateral Anti-Rotation Gripper,” sold by Skil-Care Corporation as product number 503098.

A limb stabilizing device according to one embodiment comprises a base section and one or more side sections attached to the base section and extending therefrom to define a one- or two-sided channel. The base and side section(s) define a channel exterior that resists rotation of the stabilizing device when contacting a bed or chair surface or other supporting surface. At least one of the side sections includes at least one surface discontinuity in the interior side thereof adapted to engage a limb or device received in the channel and resist rotation thereof relative to the stabilizing device.

According to some embodiments, at least one of the side sections includes at least one edge extending along the side portion adapted to engage a limb or health care device received in the channel and resist rotation thereof relative to the stabilizing device.

According to some embodiments, at least one of the side sections includes a plurality of edges extending therealong adapted to engage a limb or device received in the channel and resist rotation thereof relative to the stabilizing device.

A limb stabilizing device according to one embodiment comprises a base section and two side sections attached to the base section and extending therefrom to define a channel. Each side section includes a plurality of edges extending longitudinally along an interior side thereof

### BRIEF DESCRIPTION OF THE DRAWINGS

FIG. 1 is a perspective view of an embodiment of a bi-lateral stabilizing device.

FIG. 2 is a side elevation view of the stabilizing device depicted in FIG. 1.

FIG. 3 is side elevation view of the stabilizing device depicted in FIG. 1.

FIG. 4 is a top plan view of the stabilizing device depicted in FIG. 1.

FIG. 5 is a bottom plan view of the stabilizing device depicted in FIG. 1.

FIG. 6 is cross-section view of the stabilizing device depicted in FIG. 1 taken along line 6-6 in FIG. 1.

FIG. 7 is a perspective view of the stabilizing device depicted in FIG. 1 showing a heel boot received therein.

FIGS. 8 and 9 are perspective views similar to FIG. 1 of other embodiments of a stabilizing device similar to the stabilizing device depicted in FIG. 1, but including different numbers of longitudinally extending edges in side sections of the device.

FIG. 10 is a side elevation view of an embodiment of a unilateral stabilizing device.

FIG. 11 is a perspective view of another embodiment of a stabilizing device which includes one side compared to the stabilizing device depicted in FIG. 1 which includes two sides.

FIGS. 12 and 13 are side elevations views of a side section of stabilizing devices according to other embodiments showing two profiles of fingers on the inside of the respective device which present an edge or edges to resist rotation of a health care device in the stabilizing devices. Although the profiles are depicted as fingers, they could extend for substantial distances or the entire side of a stabilizing device.

FIGS. 14A-14G are side elevation views of stabilizing devices according to other embodiments showing various inside profiles which present an edge or edges to resist rotation of a health care device in the stabilizing devices.

### DETAILED DESCRIPTION

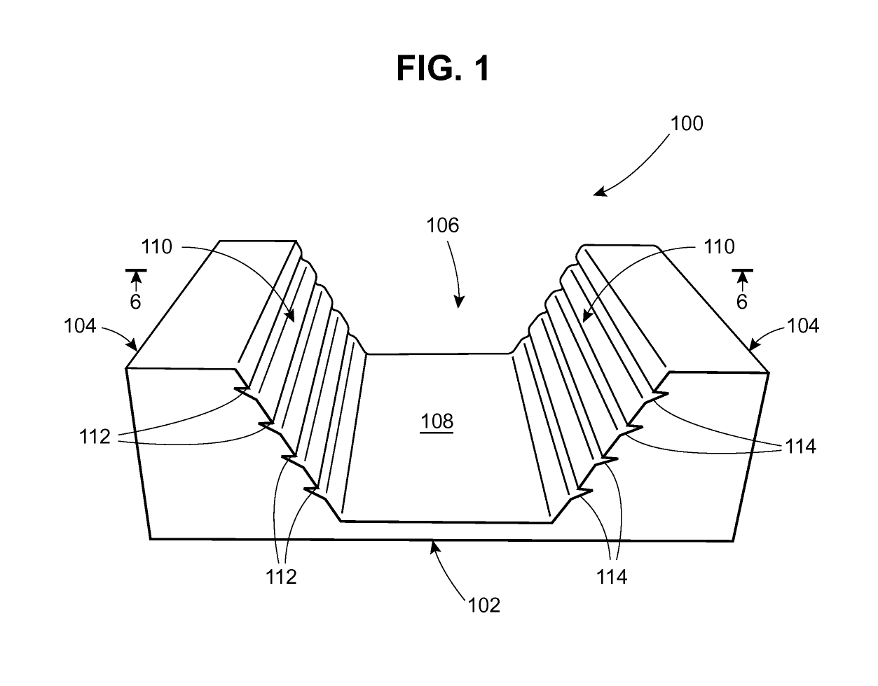

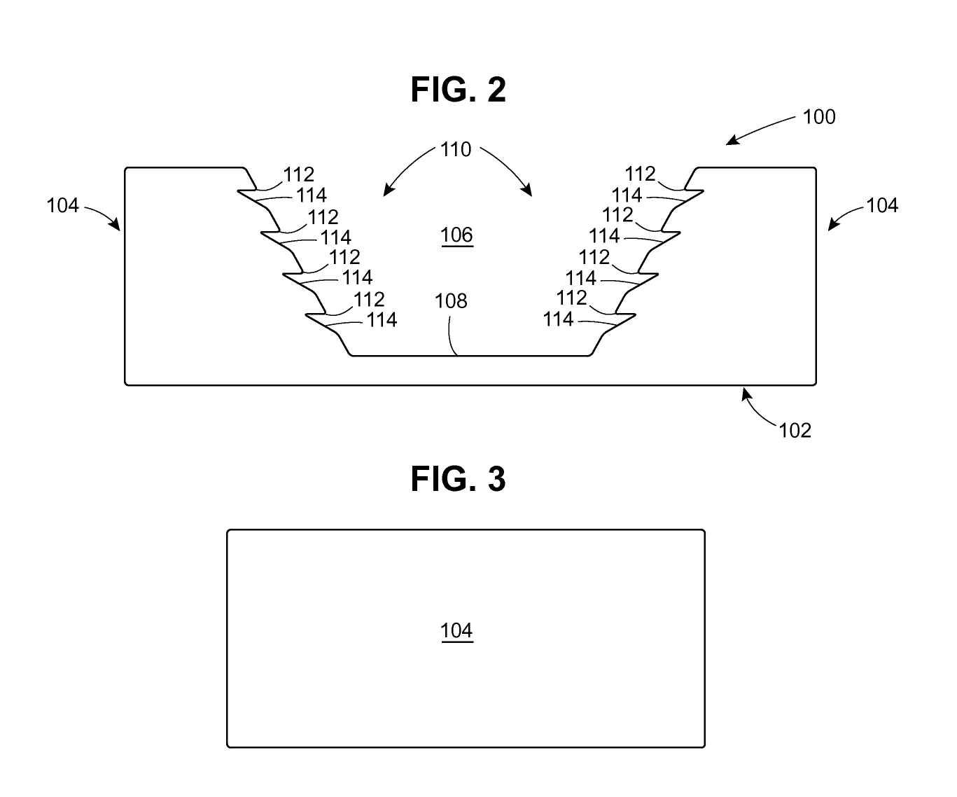

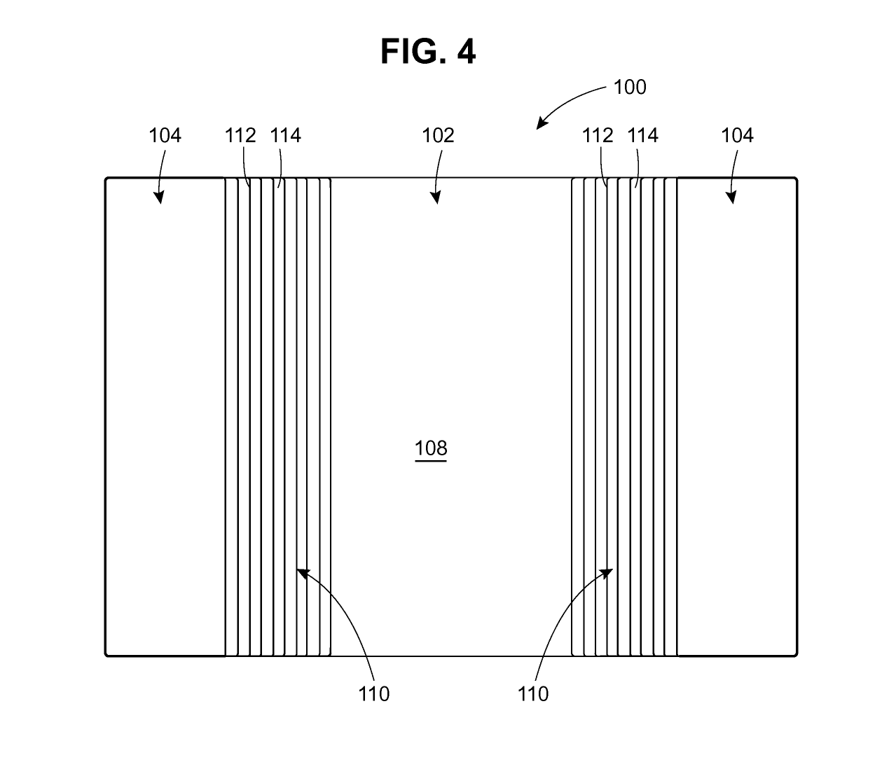

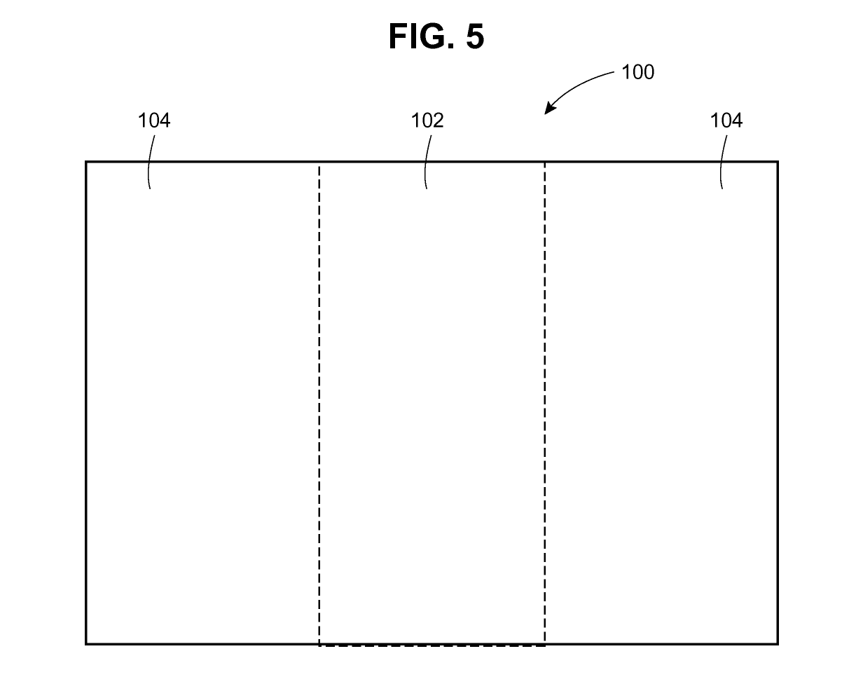

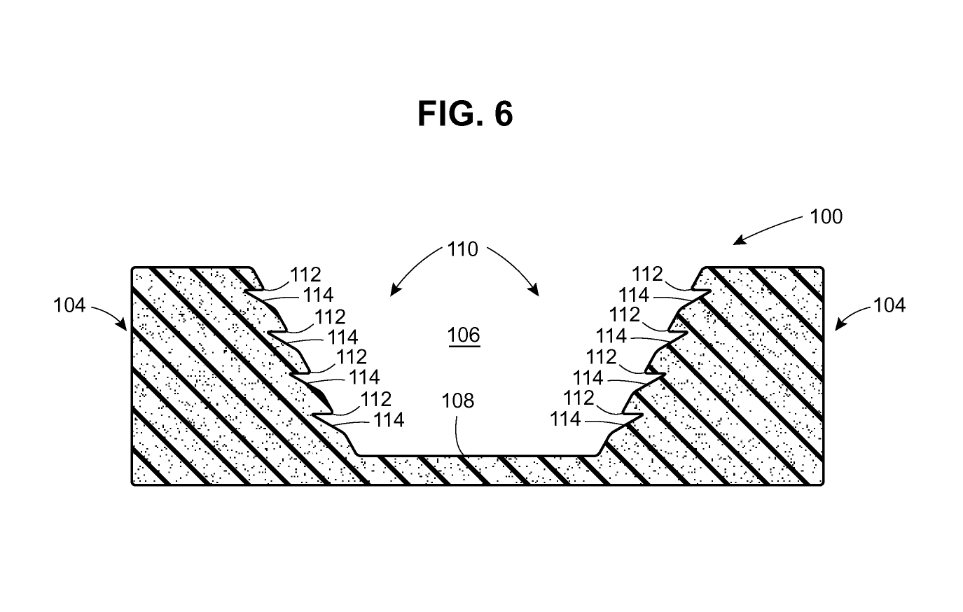

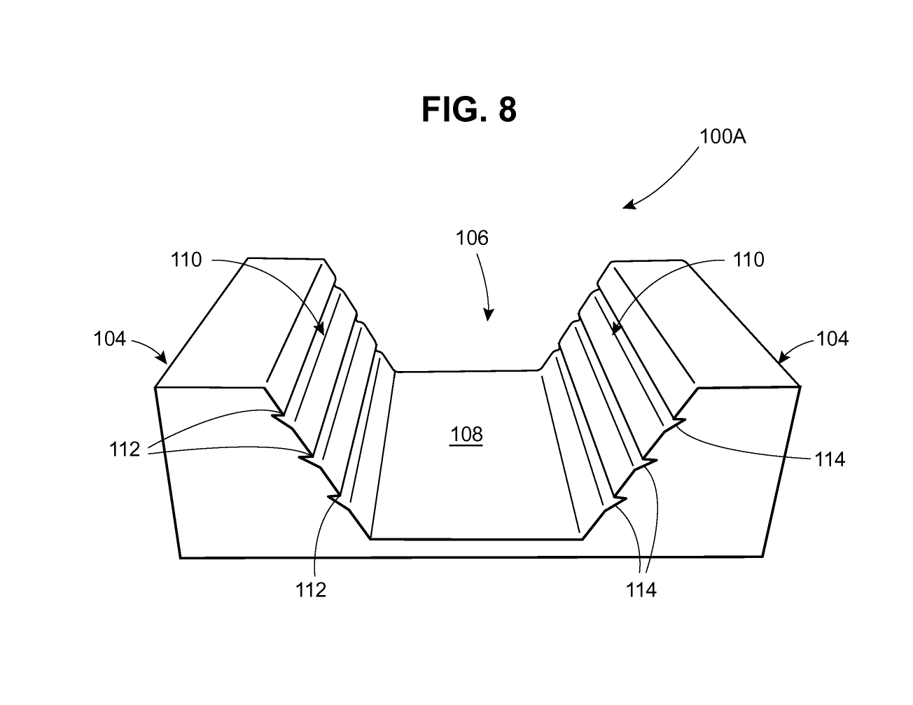

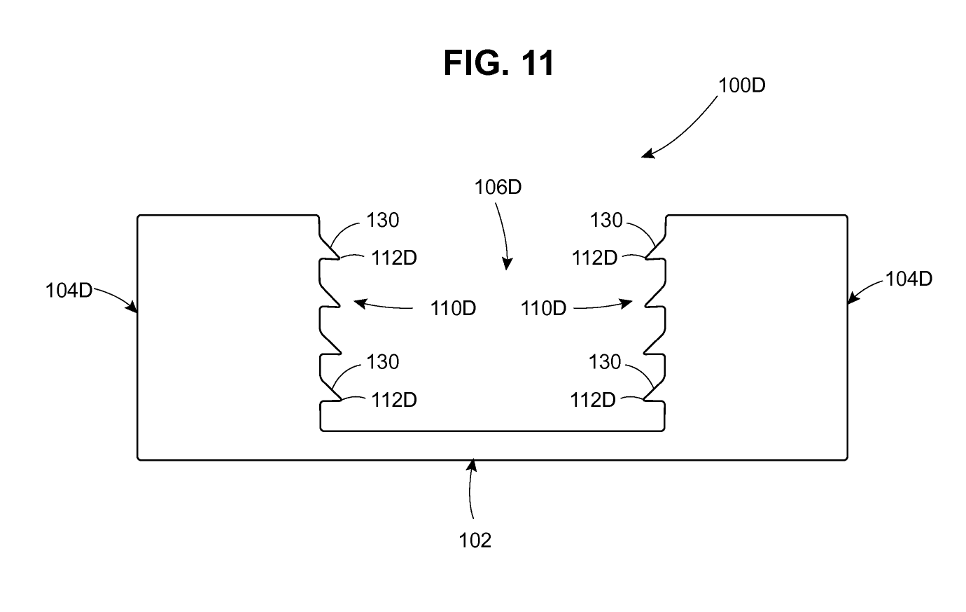

One embodiment of a stabilizing device 100 is depicted in FIGS. 1-7. Stabilizing device 100 comprises a bottom section 102, and opposed side sections 104. The bottom section 102 and the side sections 104 define a channel 106 comprising a bottom 108 and opposed sides 110. The stabilizing device 100 is configured and sized to accept a limb or health care device in the interior of the channel 106, e.g., as depicted in FIG. 7. Stabilizing device 100 provides for bi-lateral anti-rotation resistance of a limb of health care device received in the stabilizing device (as shown in FIG. 7). In the embodiment depicted in FIGS. 1-7, the side sections are wedge-shaped, but can have other shapes, e.g., block-shaped as depicted in FIG. 11, or other shapes.

A stabilizing device 100 may be of varying size, depending upon intended use. For example, the side sections may be of varying heights, either the same height for each side or different heights. Also, the bottom section may be of varying length and/or width and/or thickness.

In the embodiment of stabilizing device 100 illustrated in FIGS. 1-7, the bottom section 102 comprises a sheet, preferably flat on both the interior and exterior sides thereof, but need not be and can be contoured or shaped depending upon factors such as the particular health device used with the stabilizing device or limb, or the environment in which the stabilizing device may be used. The thickness of the sheet may also vary depending on such factors. The bottom section 102 may be flexible or inflexible to accommodate, e.g., the surface underneath the stabilizing device 100, such as an uneven bed surface, sheets, or linens. The interior and/or exterior surfaces of the bottom section may have properties that increase friction between the interior of stabilizing device and a limb or health care device, and/or between the exterior of the stabilizing device and a supporting surface such as a bed, e.g., to resist sliding of the stabilizing device.

In the embodiment of stabilizing device 10 depicted in FIGS. 1-7, the interior sides 110 include discontinuities therein, which act to engage or grip a limb or health care device received in the interior of channel 106 and thereby resist rotation thereof within the channel. The discontinuities may take various shapes and configurations. According to various embodiments, a discontinuity may comprise a discrete structure such as a spike or tab of limited width, or a more continuous structure such as an edge.

In one embodiment, as depicted in FIGS. 1, 2, 4 and 6, a discontinuity comprises an edge 112, which may be defined in various ways. For example, an edge may be formed by a recess or cut-out 114 in the interior side of a sidewall section. In another embodiment, an edge may be formed by a projection extending from the interior side of a side section. Such edges may comprise various shapes and profiles, for example, as depicted in FIGS. 1, 11-13 and 14A-14G. Regardless of the particular edge shape or profile, the edges should engage a limb or health care device in the channel so as to resist rotation thereof in the channel.

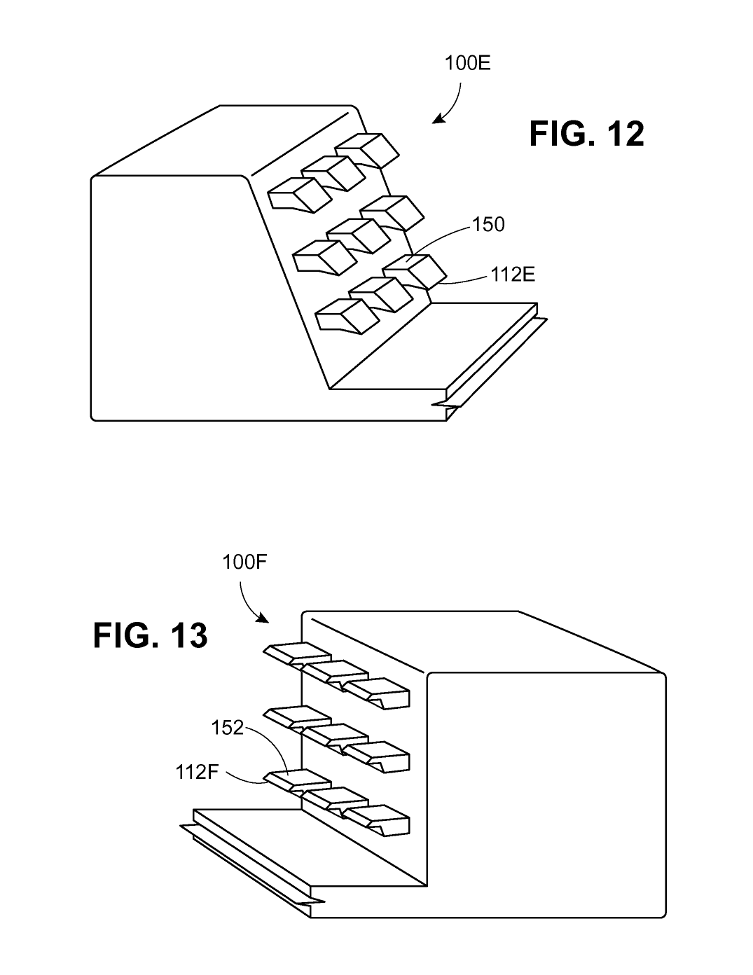

In the embodiment of the stabilizing device depicted in FIGS. 1-7, the interior side of each side portion comprises a plurality of edges 112 which each extend longitudinally therealong (relative to the channel). According to various embodiments, the edges may extend for a minor or major part of side portion. The edges 112 may be longitudinally continuous (as shown in FIG. 1), or discontinuous (with gaps therebetween), e.g., as depicted in FIGS. 12 and 13. In the embodiment depicted in FIGS. 1-7, the edges 112 are continuous and extend for the entire length of the side portion.

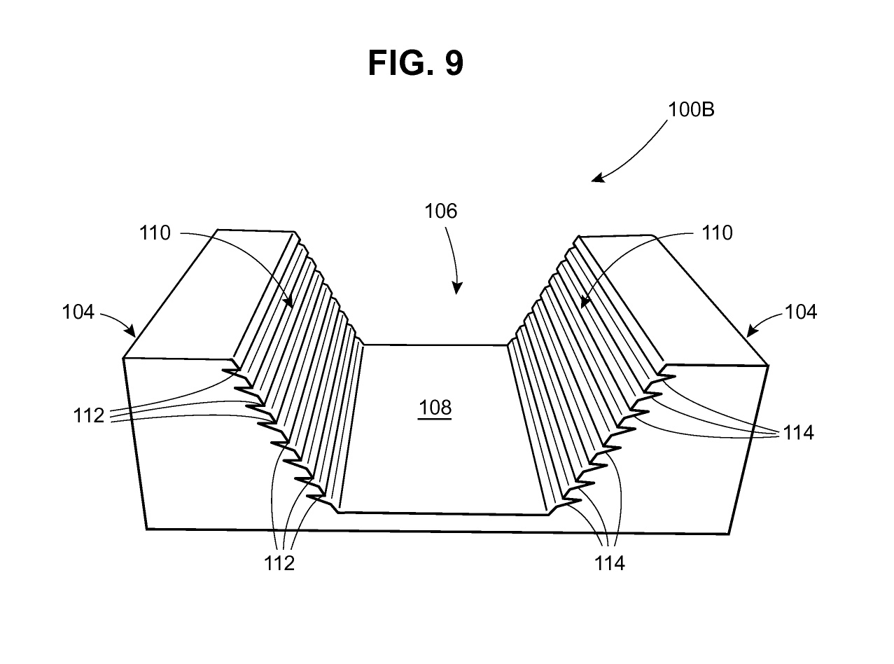

In various embodiments different levels of edges or discontinuities may be employed. For example, in the embodiment depicted in FIGS. 1-7, four levels or steps are provided, while other embodiments a greater or lesser number of levels or steps may be provided, as shown, for example in the embodiments 100A and 100B depicted in FIGS. 8 and 9, respectively, where three and seven levels or steps are provided. Spacing between levels or steps may vary from stabilizing device to stabilizing device, and within a stabilizing device. In the embodiments depicted in FIGS. 1, 8 and 9, the spacing is uniform with the respective stabilizing device, but may be different. Spacings of varying thicknesses or distance may be provided depending on, e.g., a desired engagement or grip strength or the size and/or material of the intended health care device. Edges 112 need not extend parallel to the bottom section, and may be angled toward an end or the ends of the stabilizing device to provide different effects.

In some embodiments, a recess or cavity is provided adjacent an edge, which may be of varying depth depending upon consideration of various factors discussed herein.

A stabilizing device may be configured to receive or accept a health care device used to prevent or treat conditions such as may result from confinement to a bed. For example, the stabilizing device 100 depicted in FIGS. 1-6 is configured to receive a heel boot 120 as shown in FIG. 7. It will be appreciated, however, that various health care devices may be used in combination with a suitably configured stabilizing device, and that such health care devices may configured for the foot, leg, arms, elbows, wrists, or other rotatable body parts (hereinafter referred to interchangeably with, or simply as, a “limb”). A heel boot 120 may be used to off-load, elevate, or “float” a patient's heel from a bed by suspending it over an air cavity, or otherwise lift a patient's heel or heels from a surface, such as a bed surface or bed linens (not shown). A heel boot may be secured to a patient's limb 122 in any suitable way, e.g., by straps or loops 124 closing the boot around a patient's limb. It will be appreciated that any combination, size, or placement of straps and loops may be used to secure the closure.

As mentioned, edges or other discontinuities may be configured to provide various engaging or gripping strengths or powers sufficient to prevent a health care device or limb seated in the channel 106 from rotating, e.g., to the left or the right for a bi-lateral stabilizing device, while permitting easy separation of a limb or health care device from the stabilizing device, e.g., lifting a limb while in the stabilizing device will result in such separation, per se, or with minor assistance.

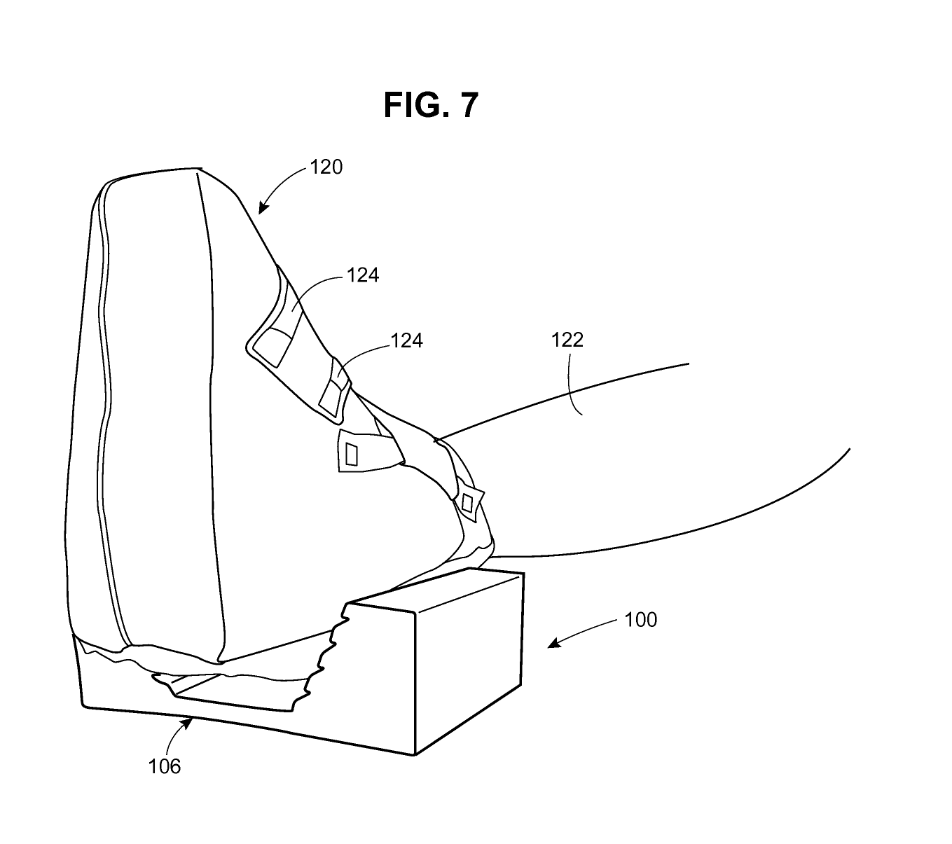

Embodiments of stabilizer devices may comprise various materials suitable structurally, and for use in various settings. For example, a stabilizer device may comprise any material suitable in a health care setting, such as in a hospital or nursing home. A stabilizer device may be made as a unitary structure, which may also be homogeneous, as illustrated in FIG. 6. In some embodiments, the stabilizer device is made of a foam material, as also illustrated in cross section in FIG. 6. The foam material may comprise a compressible foam material in some applications, or a more rigid plastic material in other applications. The material for a stabilizer device may be chosen based on desired properties such as flexibility; anti-allergenic, anti-bacterial, or anti-germicidal properties; or anti-flammability or fire retardant properties. A stabilizer device may also be made resistant to fluid, stains, or odors. In some embodiments, a stabilizer may comprise material such that the device may be considered as single use or disposable device, while in other embodiments a stabilizing device may comprise material suitable for laundering and re-use of the device.

In various embodiments, the interior sides of the side portions, or the edges or other discontinuities, may be made of a material which resists slippage of a health care device or limb. For example, edges 112, or the interior sides, or the entire stabilizing device may be made of a compressible foam material. The material of edges 112 may have properties which increase friction between the edges and a heath care device to support anti-rotation and provide increased stabilization. For example, a stabilizing device may be configured via various discontinuity configurations and materials to provide for different levels of anti-rotation. Health care providers may, as an example, wish to allow for differing degrees of internal and external hip rotation.

In various embodiments, the exterior surface of the bottom section 102 may comprise the same material as for other surfaces of the stabilizer device or a different material or materials. For example, bottom surface 102 may comprise compressible foam with any of anti-allergenic, anti-bacterial, or anti-germicidal properties, or anti-flammability or fire retardant properties. The exterior of the bottom section 102 may also comprise a material selected based on use of the device on a particular surface. For example, in various embodiments, a bottom exterior surface intended to be used in a bed setting may comprise a foam material of a certain coefficient friction to allow for a desired level of movement or restriction of movement of the stabilizer device against bed sheets, linens, etc.

According to some embodiments, the material of a stabilizer device, such as a compressible foam, may differ from the material of a health care device such as a heel boot, which may be a soft, open weave fabric with pillow style cushioning to, e.g., minimize shearing and friction, or other materials suitable for a particular patient or patient needs.

According to one embodiment, the stabilizer comprises a single piece of compressible foam, with uniform texture. In an embodiment, the stabilizer may be manufactured using a technique to allow for precision cutting of the foam material, particularly with respect to the edges and other discontinuities.

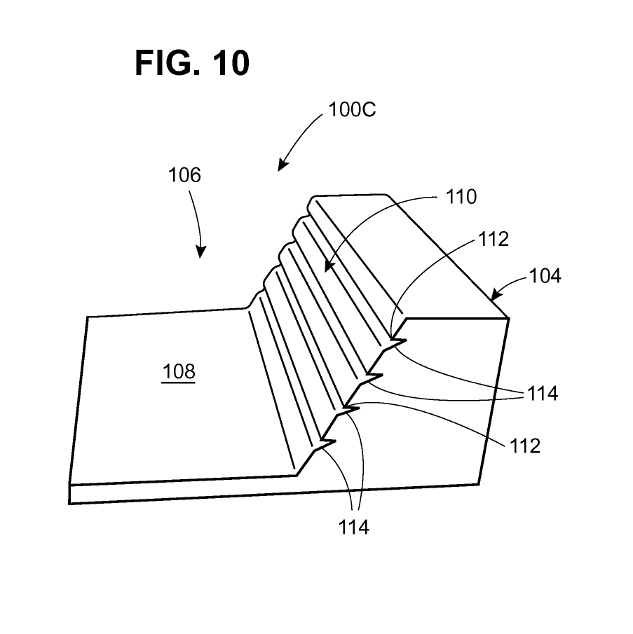

FIG. 10 depicts an embodiment of a stabilizing device 100C with a single side section 102. The description above relating to the bottom section, the side sections, the discontinuities, the materials used, the characteristics, etc., applies to stabilizer 100C as appropriate. Stabilizer 100C is intended for use in applications resisting unilateral rotation of a limb and/or health care device.

The stabilizer device 100D depicted in FIG. 11 comprises side sections 104D which are block-shaped. The side sections 104D and the bottom section 102 form a channel 106D which is rectangularly shaped. The interior sides 110D comprise discontinuities defined by projections 130 which comprise edges 112D. Similar to the edges 112 in the embodiment of FIGS. 1-6, edges 112D may, but need not, extend for the length of the sides 110D. The discussion above, including that regarding discontinuities, generally applies to the embodiment of FIG. 11.

As mentioned above, the discontinuities in the interior side portions may take varying sizes, shapes, profiles, contours, etc. FIGS. 12, 13 and 14A-G are meant to illustrate a few non-limiting examples. In some embodiments, a discontinuity may be embodied by a finger or tab 150, 152, as illustrated in FIGS. 12 and 13, which present an edge or edges as described above. However, the width of a finger may vary from that of a finger or tab, to a minor, major or the entire extent of the stabilizing device.

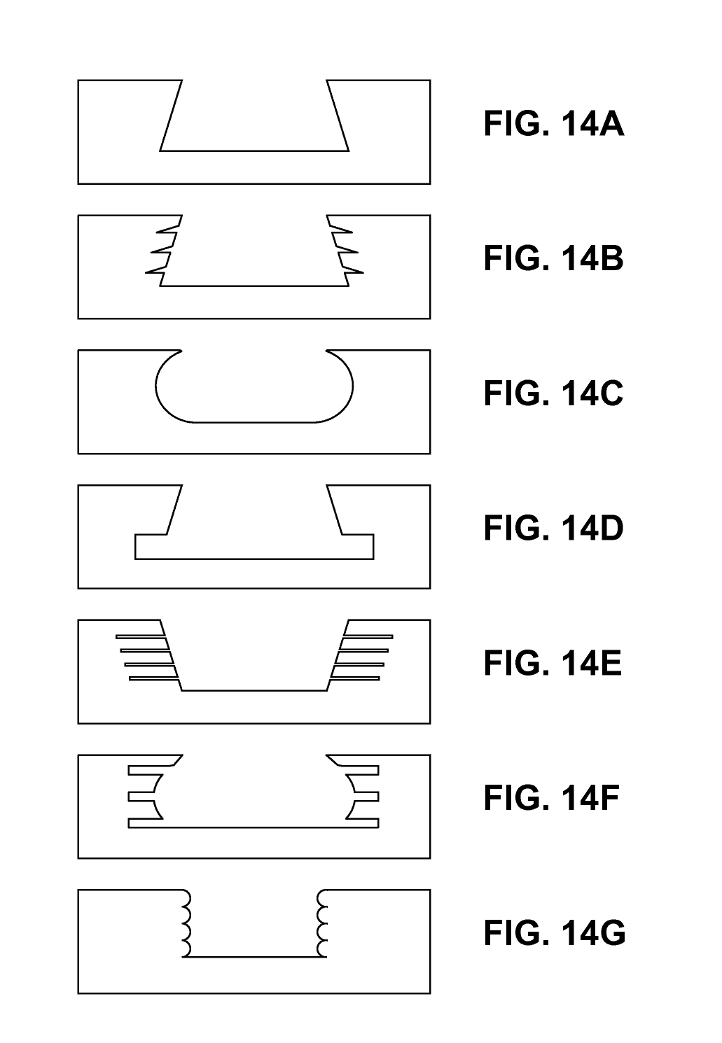

FIGS. 14A-G show embodiments of more profiles of structure providing a discontinuity or discontinuities and an edge or edges to resist rotation of a health care device within the stabilizing device. As discussed in connection with FIGS. 12 and 13, the width of such structure may vary from the entire extent of the stabilizing device down to a finger or tab.

The profile of a projection embodying a discontinuity in some embodiments may be beveled on one side to provide a camming effect to entry of a health care device into the channel of the stabilizing device, while the edge of the projection may face downwardly into the channel or be squared to improve an engaging or gripping function to resist rotation. In some embodiments, a projection or edge may be profiled to provide or improve engagement or grip in two directions.

While the invention has been described and illustrated in connection with illustrative embodiments, many variations and modifications as will be evident to those skilled in this art may be made without departing from the scope of the invention as defined by the claims, and the invention is thus not to be limited to the precise details of methodology or construction set forth above as such variations and modifications are intended to be included within the scope of the invention as defined by the claims.

What is claimed is:

1. A bi-lateral anti-rotation wedge comprising:
a base section; and
two side sections attached to the base section and extending at an angle therefrom to define a trough-shaped channel;
wherein each side section includes a plurality of edges extending longitudinally along an interior side thereof
